(19) United States
(12) Patent Application Publication
(10) Pub. No.: US 2024/0308955 A1
(43) Pub. Date: Sep. 19, 2024

(54) METHOD FOR SYNTHESIZING AMORPHOUS NOBLE METAL-CRYSTALLINE SEMINCONDUCTOR/METAL HETEROPHASE NANOPARTICLES

(71) Applicant: City University of Hong Kong, Hong Kong (HK)

(72) Inventors: Hua ZHANG, Hong Kong (HK); Li ZHAI, Hong Kong (HK); Biao HUANG, Hong Kong (HK)

(21) Appl. No.: 18/184,688

(22) Filed: Mar. 16, 2023

(51) Int. Cl. C07C 249/02; B01J 23/44; B01J 23/50; B01J 23/52; B01J 27/04; B01J 27/043; B01J 35/00; B01J 37/02; B01J 37/03; B01J 37/04

(52) U.S. Cl. CPC C07C 249/02; B01J 23/44; B01J 23/50; B01J 23/52; B01J 27/04; B01J 27/043; B01J 35/23; B01J 35/39; B01J 37/0221; B01J 37/035; B01J 37/04

(57) ABSTRACT

A robust and general method is provided to synthesize noble metal-based amorphous-crystalline heterophase nanoparticles, each having an amorphous noble metal core and a crystalline semiconductor/metal shell or a Janus structure with an amorphous noble metal domain and a crystalline metal domain attached side by side with the amorphous noble metal domain (i.e., snowman-like structure). The as-synthesized heterophase nanoparticles not only exhibit superior activities in diverse catalytic reactions but also show unexpected high stability, which could be used as ideal templates for the seeded growth of other nanostructures, thus show tremendous potential in different applications including electrocatalysis and photocatalysis. With efficiently separated photo-induced electron and photo-induced holes, superior catalytic performance of amorphous nanomaterials, efficient solar energy conversion ability of crystalline semiconductors, as well as the synergistic effect between them, the controlled construction of amorphous noble metal-crystalline semiconductor heterostructures can be a promising route to development of high-performance catalysts towards photocatalytic reactions.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention generally relates to synthesis of amorphous-crystalline semiconductor heterophase nanoparticles. More specifically the present invention relates to well-controlled, robust and general synthesis of amorphous noble metal-crystalline semiconductor/metal heterophase nanoparticles.

### BACKGROUND OF THE INVENTION

Heterostructure nanomaterials have attracted tremendous attention due to their unique physicochemical properties arising from the diverse and tunable morphologies, interfaces, and spatial arrangements of different components in the heterostructures. These nanomaterials could exhibit superior performance over their individual components in a wide range of applications due to the synergistic effect between the different components. Among various heterostructures, noble metal-based heterostructures have emerged as important members. Benefiting from the excellent intrinsic catalytic properties of noble metals, tunable bandgaps, as well as the electronic interactions between them, the noble metal-based heterostructures constructed via rationally integrating noble metal and other nanostructures, such as semiconductor, non-noble metals could exhibit superior performance in various applications, especially in catalysis. For example, Pt nanoparticles are reported to be controllably grown on the tip of CdS nanorods to form Pt-CdS heterostructures as a highly efficient photocatalyst for overall water splitting into H₂ and O₂. Importantly, the time-resolved photoluminescence spectra and ultrafast transient absorption revealed that the photoelectrons generated by CdS could be trapped by Pt nanoparticles, which enabled the efficient water reduction reaction at Pt sites. Notably, rational design and growth of a series of noble metals (Ag, Pd, Pt) on MoS₂ nanosheets have been realized to form 0D-2D noble metal-semiconductor heterostructures. Detailed characterizations found that the noble metal nanoparticles are epitaxially grown on the MoS₂ nanosheets. Impressively, the prepared Pt-MoS₂ heterostructures showed superior catalytic activity for HER, much better than the pure MoS₂ and commercial Pt catalyst at the same Pt loading.

In the past few decades, tremendous efforts have been devoted into finely tuning the morphologies, interfaces, and spatial arrangements of different components in the noble metal-based heterostructures to achieve better performance in various applications. Recently, in addition to the aforementioned features, crystal phase has been emerging as an important structural parameter that determines the properties and functionalities of nanomaterials. Previous studies revealed that noble metals with unconventional phases could exhibit intriguing physicochemical properties and enhanced catalytic performance. In particular, noble metal nanomaterials with long-range disordered structure, i.e., amorphous structure, have emerged as highly efficient catalysts for various reactions due to the abundant uncoordinated sites and dangling bonds as compared with their crystalline counterparts. For example, the phase transformation of face-centered cubic (fcc) Pd nanomaterials into amorphous Pd has been achieved via ligand exchange. Importantly, the obtained amorphous Pd nanoparticles exhibited much higher HER activity than the crystalline fcc Pd. In addition, ultrathin Rh and Rh-based alloy nanosheets are also prepared, which exhibited enhanced catalytic activity and selectivity toward the direct synthesis of indole compared to their crystalline counterparts.

Hence, by virtue of the superior catalytic performance of amorphous noble metal nanomaterials, the efficient solar energy conversion ability of semiconductors, as well as the synergistic effect between them, it is believed that the controlled construction of amorphous noble metal-crystalline semiconductor heterostructures could be a promising route to the development of high-performance catalysts towards photocatalytic hydrogen evolution reactions. However, till now, the precise control over the phases of metal-semiconductor heterostructures has rarely been achieved, thus severely hindering the understanding of the structure-property relationships and phase-dependent applications. The challenge mainly comes from the metastable nature of the noble metal nanomaterials with unconventional phases, i.e., these noble metal nanostructures would transform into their thermodynamically stable phase after the growth of semiconductors.

Imines are very important and highly valuable chemicals that are crucial for manufacturing pharmaceuticals, pesticides, biologically active compounds, fragrances and dyes, especially N-benzylbenzaldimine. In the conventional process, the synthesis of N-benzylbenzaldimine mainly relies on the condensation reaction between amines and carbonyl groups, which require a dehydrating agent, aldehydes, and Lewis acids as primary sources to drive the reaction at high temperature. The above-mentioned synthesis process commonly needs high pressure of H₂ and high temperature, which is expensive and dangerous. Thus, the approach to obtaining imine through direct photocatalytic reactions under mild reaction conditions using abundant solar energy is promising. However, it is hard to obtain pure N-benzylbenzaldimine using photocatalytic reactions due to the further hydrogenation of N-benzylbenzaldimine into other products. Currently, some homogeneous photocatalyst could be used to achieve a high-selectively synthesis of N-benzylbenzaldimine. However, these homogeneous catalysts are hard to be separated from the N-benzylbenzaldimine, which greatly hinders their real applications and prevents us from recycling the catalysts.

### SUMMARY OF THE INVENTION

In accordance with a first aspect of the present invention, a method for synthesizing amorphous noble metal-crystalline semiconductor heterophase nanoparticles is provided. Each amorphous noble metal-crystalline semiconductor heterophase nanoparticle has an amorphous noble metal core and a crystalline semiconductor shell. The provided method comprises: mixing amorphous noble metal-based nanoparticle seeds, chalcogen and a first solvent to form a first mixture; mixing a metal precursor, fatty acid and a second solvent to form a second mixture; degassing the second mixture at a degassing temperature for a degassing time under magnetic stirring; heating the second mixture to a first temperature under nitrogen (N₂) atmosphere; cooling the second mixture to a second temperature; injecting the first mixture into the second mixture to form third mixture; keeping the third mixture at a growth temperature for a growth time to form the noble metal-based heterophase nanoparticles.

In accordance with one embodiment of the first aspect of the present invention, the amorphous noble metal-based nanoparticle seeds are amorphous palladium (Pd)-based nanoparticle seeds; the chalcogen is ammonium thiocyanate (NH₄SCN); and a weight ratio of the amorphous Pd-based nanoparticle seeds to the NH₄SCN is 1:3.

In accordance with another embodiment, the metal precursor includes one or more cadmium (Cd)-based compounds such that the core is constructed of amorphous Cd and the shell is constructed of crystalline cadmium sulphide (CdS).

In accordance with another embodiment, the one or more Cd-based compounds include cadmium oxide (CdO) and cadmium chloride (CdCl₂).

In accordance with another embodiment, weight ratios of amorphous Pd-based nanoparticle seeds to the CdO and CdCl₂ are 1:6 and 10:9 respectively.

In accordance with another embodiment, the metal precursor includes one or more nickel (Ni)-based compounds such that the core is constructed of amorphous Pd and the shell is constructed of crystalline nickel sulphide (Ni₂S₃).

In accordance with another embodiment, the one or more Ni-based compounds include nickel(II) bis(acetylacetonate) (Ni(acac)₂).

In accordance with another embodiment, a weight ratio of the amorphous Pd-based nanoparticle seeds to the Ni(acac)₂ is 1:5.

In accordance with another embodiment, the metal precursor includes one or more copper (Cu)-based compounds such that the core is constructed of amorphous Pd and the shell is constructed of crystalline copper sulphide (Cu₂-xS).

In accordance with another embodiment, the one or more Cu-based compounds include copper (II) chloride (CuCl₂).

In accordance with another embodiment, a weight ratio of the amorphous Pd-based nanoparticle seeds to the CuCl₂ is 1:5.

In accordance with a second aspect of the present invention, a synthesizing amorphous noble metal-crystalline metal heterophase nanoparticles is provided. Each amorphous noble metal-crystalline metal heterophase nanoparticle has a Janus structure with an amorphous noble metal domain and a crystalline metal domain attached side by side with the amorphous noble metal domain (i.e., snowman-like structure). The provided method comprises: dispersing amorphous noble metal-based nanoparticle seeds into a first solvent to form a first mixture; degassing the first mixture at room temperature; preheating the first mixture under nitrogen (N₂) atmosphere at a preheat temperature for a preheat time under magnetic stirring; dissolving a metal precursor in a second solvent to form a second mixture; injecting the second mixture into the first mixture to form a third mixture at a constant injection rate; keeping the third mixture at a growth temperature for a growth time to form the amorphous noble metal-crystalline metal heterophase nanoparticles.

In accordance with one embodiment of the second aspect of the present invention, the amorphous noble metal-based nanoparticle seeds are amorphous palladium (Pd)-based nanoparticle seeds.

In accordance with another embodiment, the metal precursor includes one or more gold (Au)-based compounds such that a Janus structure with an amorphous noble metal domain and a crystalline metal domain attached side by side with the amorphous noble metal domain (i.e., snowman-like structure) is obtained.

In accordance with another embodiment, the one or more Au-based compounds include hydrogen tetrachloroaurate(III) (HAuCl₄·xH₂O).

In accordance with another embodiment, a weight ratio of the amorphous Pd-based nanoparticle seeds to the HAuCl₄·xH₂O is 1:5.

In accordance with another embodiment, the metal precursor includes one or more silver (Ag)-based compounds such that a Janus structure with an amorphous noble metal domain and a crystalline metal domain attached side by side with the amorphous noble metal domain is obtained.

In accordance with another embodiment, the one or more Ag-based compounds include silver nitrate (AgNO₃).

In accordance with another embodiment, a weight ratio of the amorphous Pd-based nanoparticle seeds to the AgNO₃ is 1:5.

In accordance with a third aspect of the present invention, a method of using amorphous Pd-crystalline CdS heterostructure nanoparticles as photocatalysts in a photocatalytic C—N coupling reaction to produce hydrogen and an imine is provided. The method comprises: dissolving the amorphous Pd-crystalline CdS heterostructure nanoparticles in an organic solvent to form a first mixture; mixing NH₄SCN in N-methylformamide to form a second mixture; dispersing the second mixture into the first mixture with vigorous stirring to transform the amorphous Pd-crystalline CdS heterostructure nanoparticles to a solid product with a N-methylformamide phase; washing the solid product with ethanol; dispersing the washed solid product in acetonitrile to form a third mixture; adding an amine into the third mixture to form a fourth mixture; degassing the fourth mixture; keeping the degassed fourth mixture at room temperature under nitrogen (N₂) atmosphere; irritating the fourth mixture with a 300 W Xe lamp to produce the hydrogen and convert the amine into the imine.

In one embodiment of the third aspect of the present invention, the amine is a benzylamine and the imine is a N-benzylbenzaldimine.

The present invention provides a facile seeded method for synthesizing noble metal nanomaterials with novel and unique heterostructures with well-defined amorphous-crystalline interfaces including noble metal-semiconductor and noble metal-noble metal heterostructures. The as-synthesized heterostructure nanoparticles not only exhibit superior activities in diverse catalytic reactions but also show unexpected high stability, which could be used as ideal templates for the seeded growth of other nanostructures, thus show tremendous potential in different applications including electrocatalysis and photocatalysis. As a typical example, the as-prepared amorphous Pd-crystalline CdS aPd-cCdS) heterostructures have exhibited superior activity and selectivity in the photocatalytic C—N coupling reactions to produce high value-added imines.

Moreover, with efficiently separated photo-induced electron and photo-induced holes, together with the superior catalytic performance of amorphous nanomaterials, efficient solar energy conversion ability of crystalline semiconductors, as well as the synergistic effect between them, the controlled construction of amorphous noble metal-crystalline semiconductor heterostructures can be a promising route to the development of high-performance catalysts towards photocatalytic reactions. As a typical example, the aPd-cCdS heterostructures are used as photocatalysts to simultaneously produce clean energy (e.g., H₂) and high value-added imines with a remarkably high evolution rate in the photocatalytic C—N coupling reactions, therefore have great applications in industrial catalysis to produce highly valued organics and clean energy.

### BRIEF DESCRIPTION OF THE DRAWINGS

Embodiments of the invention are described in more details hereinafter with reference to the drawings, in which:

FIGS. 1A to 1C illustrate transmission electron microscopy (TEM) image, high-resolution transmission electron microscopy (HRTEM) image, and selected area electron diffraction (SAED) pattern of the synthesized aPd nanomaterials.

FIG. 2A illustrates X-Ray diffraction (XRD) pattern of the synthesized aPd nanomaterials (with standard diffraction peaks of fcc-Pd as references); FIG. 2B illustrates high-resolution X-ray photoelectron spectroscopy (XPS) spectrum of Pd 3d.

FIG. 3A is a schematic illustration of the synthesis of aPd-cCdS heterostructures by using the pre-synthesized aPd nanomaterials as seeds for the growth of CdS nanorods according to one embodiment of the present invention. FIG. 3B shows high-angle annular dark-field scanning transmission electron microscopy (HAADF-STEM) of aPd-cCdS heterostructures. FIG. 3C shows spherical aberration HAADF-STEM (Cs-HAADF-STEM) image of aPd-cCdS heterostructures. FIG. 3D illustrates fast Fourier transform (FFT) pattern of selected area of aPd-cCdS in FIG. 3C. FIG. 3E illustrates HAADF-STEM images and the corresponding elemental mapping images of aPd-cCdS heterostructures.

FIG. 4A illustrates XRD pattern of the synthesized aPd-cCdS heterostructures (with standard diffraction peaks of wurtzite CdS and fcc-Pd used as references). FIGS. 4B and 4C illustrate high-resolution XPS spectrum of Pd 3d and S 3d of the aPd-cCdS heterostructures respectively. FIG. 4D illustrates normalized Pd K-edge XANES spectra of the aPd-cCdS heterostructures, aPd nanocrystals, PdS nanocrystals and Pd foil. FIG. 4E illustrates Fourier-transformed EXAFS. FIG. 4F illustrates normalized R spacing EXAFS spectra of the aPd-cCdS heterostructures, aPd nanocrystals, PdS nanocrystals and Pd foil.

FIGS. 5A and 5B illustrate photoluminescence (PL) intensity spectra and time-resolved PL spectra of aPd-cCdS heterostructures respectively. FIGS. 5C and 5D illustrates two-dimensional pseudo-color plots of pump-visible probe transient absorption (TA) spectra of the colloid solutions of CdS and aPd-cCdS heterostructures in toluene respectively. FIGS. 5E and 5F illustrate time-resolved absorption spectrum of CdS and aPd-cCdS heterostructures in toluene respectively.

FIG. 6A is a schematic illustration of the simultaneous H₂ evolution and C—N coupling reactions using CdS and aPd-cCdS heterostructures according to one embodiment of the present invention. FIG. 6B shows H₂ evolution rate of aPd-cCdS heterostructures under simulated solar light and 420 nm light. FIG. 6C shows conversion rate of benzylamine using CdS and aPd-cCdS heterostructures in photocatalytic reactions under simulated solar light and 420 nm light. FIG. 6D shows selectivity of photocatalytic benzylamine into N-benzylbenzaldimine using CdS and aPd-cCdS heterostructures under simulated solar light. FIG. 6E shows Recycling of aPd-cCdS heterostructures in photocatalytic C—N coupling reactions.

FIGS. 7A and 7B illustrate gas chromatography (GC) spectra and the corresponding mass spectrometry (MS) spectra of the substrate and product in photocatalytic C—N coupling reactions using aPd-cCdS heterostructures respectively.

FIGS. 8A and 8B illustrate proton nuclear magnetic resonance (¹H NMR) and carbon-13 (¹³C NMR) spectra of the product in photocatalytic C—N coupling reactions using aPd-cCdS heterostructures respectively.

FIGS. 9A and 9B illustrate GC spectra of the product in photocatalytic C—N coupling reactions using aPd-cCdS heterostructures and CdS respectively.

FIG. 10 shows GC spectra and the corresponding MS spectra of product in photocatalytic C—N coupling reactions using fcc Pd-cCdS heterostructures.

FIG. 11 shows HAADF-STEM and the corresponding elemental mapping images of amorphous Pd-crystalline Cu₂₋ₓS (aPd-cCu₂₋ₓS) heterostructures.

FIG. 12 shows HAADF-STEM and the corresponding elemental mapping images of amorphous Pd-crystalline Ni₂S₃(aPd-cNi₂S₃) heterostructures.

FIGS. 13A to 13C illustrate TEM image, STEM image of amorphous Pd-crystalline Au (aPd-cAu) heterostructure and high-resolution TEM image of a representative aPd-cAu heterostructure. FIG. 13D shows FFT patterns obtained from the corresponding marked area in FIG. 13C. FIGS. 13E and 13F shows SAED pattern, EDS elemental mapping of the aPd-cAu heterostructure. FIG. 13G shows EDS line scan along the white line in the STEM image in FIG. 13F.

FIGS. 14A to 14C illustrate TEM images and high-resolution TEM image of a representative amorphous Pd-crystalline Ag (aPd-cAg) heterostructure. FIG. 14D shows FFT patterns obtained from the corresponding marked area in FIG. 14C. FIG. 14E shows SAED pattern the aPd-cAg heterostructure.

### DETAILED DESCRIPTION

In the following description, embodiments of the present invention are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present invention is aimed to provide robust and general methods to synthesize noble metal-based amorphous-crystalline heterophase nanomaterials with superior and unique catalytic properties.

In the first aspect of the present invention, a robust and general wet-chemical method is provided to synthesize amorphous noble metal-crystalline semiconductor heterophase nanoparticles having an amorphous noble metal core and a crystalline semiconductor shell. The provided method comprises: mixing amorphous noble metal-based nanoparticle seeds, chalcogen and a first solvent to form a first mixture; mixing a metal precursor, fatty acid (e.g. oleic acid) and a second solvent to form a second mixture; degassing the second mixture at a degassing temperature for a degassing time under magnetic stirring; heating the second mixture to a first temperature under nitrogen (N₂) atmosphere; cooling the second mixture to a second temperature; injecting the first mixture into the second mixture to form third mixture at a constant injection rate; keeping the third mixture at a growth temperature for a growth time to form the noble metal-based heterophase nanoparticles.

In accordance with some embodiments of the present invention, the provided method is used for synthesizing amorphous Pd (aPd)-crystalline semiconductor heterophase nanoparticles and comprises the preparation of aPd nanoparticles as templates (or seeds), usage of oleylamine, fatty acid (e.g., oleic acid), and 1-octadecene as solvents, metal-based compounds (such as Cd, Cu or Ni-based compound) as metal precursors, as well as NH₄SCN as chalcogen precursors. Through the hot injection of chalcogen precursors (NH₄SCN in oleylamine) into reactors containing metal precursors and aPd seeds at a relatively low temperature. It is demonstrated that amorphous nature of the aPd seeds is maintained and crystalline CdS (cCdS), crystalline Cu₂₋ₓS(cCu₂₋ₓS), crystalline Ni₂S₃(cNi₂S₃) are successfully grown on aPd seeds to form aPd-cCdS, aPd-cCu₂₋ₓS, aPd-cNi₂S₃ heterophase nanoparticles respectively.

### Synthesis of aPd Nanomaterials

In a typical synthesis of aPd nanomaterials, 20 mg of Pd(acac)₂, 240 μL of Tri-n-octylphosphine, 1 mL of oleic acid, 20 mL of oleylamine, 10 mL of 1-octadecene are added into a 50 mL three-neck flask to form a mixture. The mixture is degassed upon heating at 80-120° C. under vacuum with vigorous magnetic stirring for 10 minutes. Then, the mixture is purged with Ar and heated to 280-320° C. and maintained at the temperature for 1 hour. The obtained aPd are collected by centrifugation at 8000 rpm for 3 min, and then dispersed in a 10 mL mixture of organic solvents (e.g., toluene and ethanol (v/v=6/4)). The obtained aPd is collected by centrifugation at 8000 rpm for 3 min, and then redispersed in 10 mL organic solvent (e.g., toluene) for storage.

### Characterization of aPd Nanomaterials

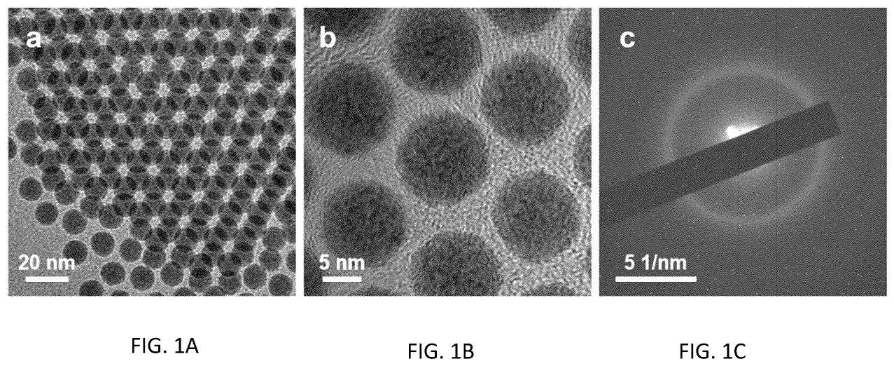

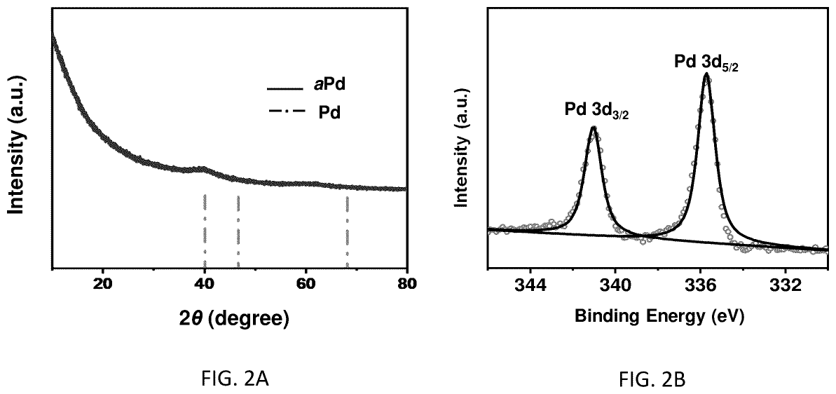

The TEM image (FIG. 1A) and HRTEM image (FIG. 1B) show that there are no crystalline parts in the aPd nanocrystals. The synthesized aPd nanoparticles have uniform sizes and possess an average size of ˜10 nm. The amorphous nature is further confirmed by the diffuse ring in the SAED pattern (FIG. 1C) and a weak and broad peak near 40.1° in the XRD pattern of aPd (FIG. 2A). XPS) is used to determine the valence state of components in aPd. In particular, the peaks located at 335.6 and 340.8 eV can be ascribed to Pd 3d3/2 and Pd 3d5/2 of zerovalent Pd, respectively (FIG. 2B). Combined XPS with HRTEM and XRD results, it can be concluded that the amorphous Pd nanocrystals with uniform size have been successfully prepared.

Synthesis and Characterization of aPd-cCdS Heterophase Nanoparticles

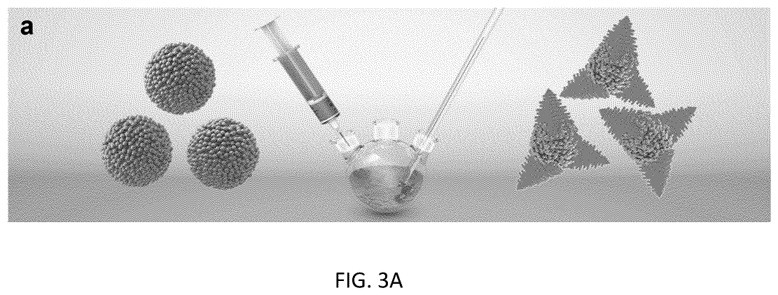

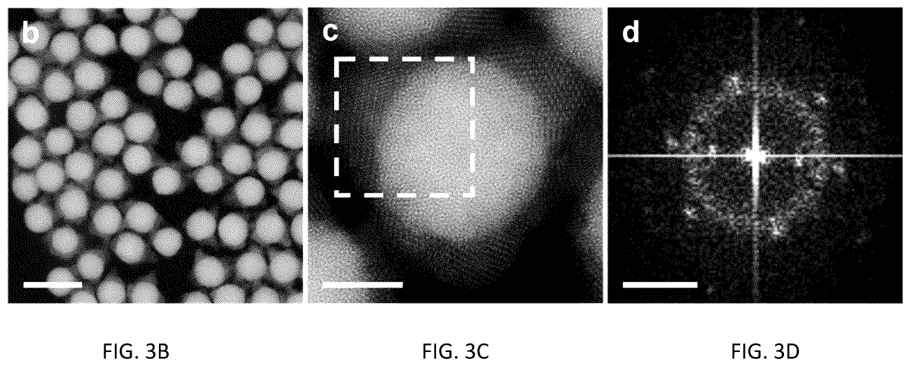

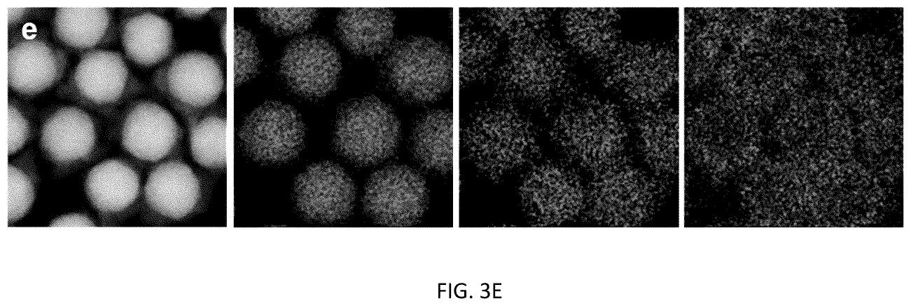

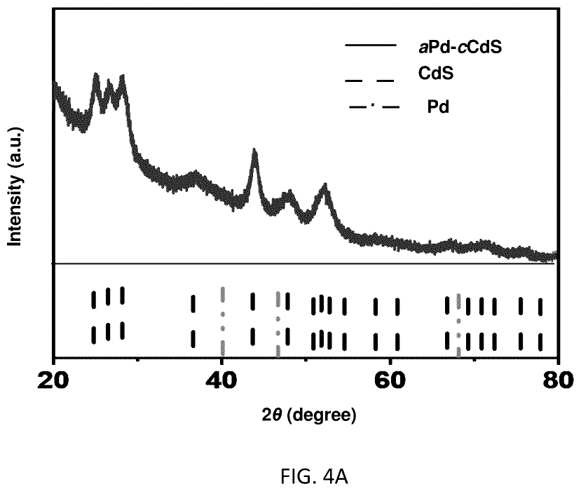

In a typical synthesis of aPd-cCdS, 12 mg of CdO, 1.8 mg of CdCl₂, 1.75 mL of oleic acid, 6 mL of 1-octadecene are added into a 50 mL three-neck flask (FIG. 3A) and degassed at 100° C. for 30 min under vigorous magnetic stirring at 750 r.p.m. After it is heated to 260° C. under N₂ atmosphere, the solution becomes transparent. The solution is cooled to 200° C., and a mixture of 2 mg of aPd seeds, 6 mg of NH₄SCN, 1.5 mL of an organic solvent (e.g., oleylamine) is injected into the flask in a constant injection rate ranging from 1 to 10 mL/h, and the temperature is kept at 190-200° C. for 2 to 6 min. Then the reaction is stopped by removing the heating mantle. After the solution is cooled down to 100° C., 5 mL of an organic solvent (e.g., toluene) is injected into the reaction flask. Then, 5 mL of ethanol is added to the solution, and the product is collected by centrifuge at 8,000 r.p.m. for 3 min. The obtained precipitate is washed with the mixture of organic solvents (e.g., toluene and ethanol (with a volume ratio v/v=1/1)) and then dispersed into 10 mL of an organic solvent (e.g., toluene).

The HAADF-STEM image shows that the synthesized amorphous Pd-crystalline CdS have multi-branched structures with uniform sizes of CdS nanorods on amorphous Pd (FIG. 3B). As shown in the Cs-HAADF-STEM image (FIG. 3C), the amorphous structure of Pd NPs could be well maintained after the growth of CdS. The FFT pattern clearly revealed the amorphous nature of the core materials and the crystalline nature of shell materials (FIG. 3D). The energy dispersive X-ray spectrometry (EDS) mappings demonstrate that Cd and S elements constructed homogeneous shells on the amorphous Pd NPs (FIG. 3E).

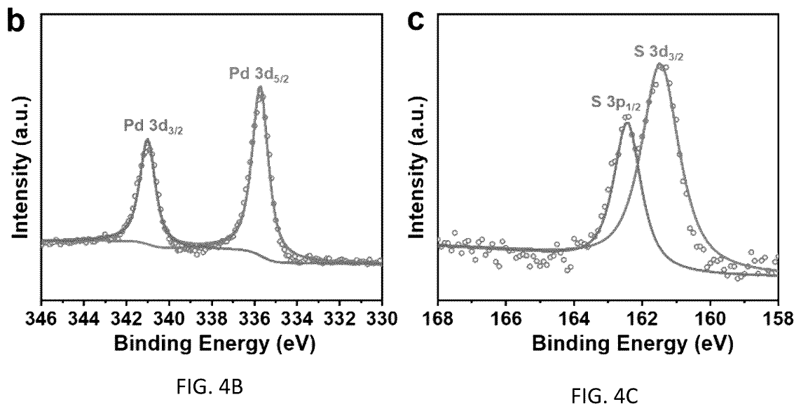

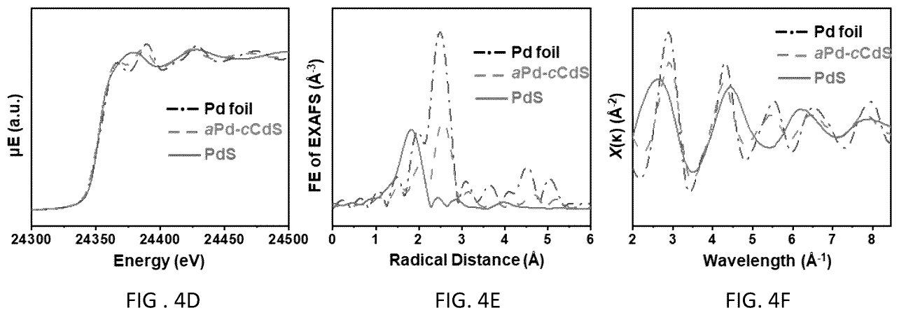

Moreover, no diffraction peak of crystalline Pd is found in the XRD curve of aPd-cCdS (FIG. 4A), the peaks located at 24.9°, 26.5°, 28.2°, 36.6°, 43.7°, 47.8° and 51.9° are well matched with the (100), (002), (101), (102), (110), (103) and (112) planes of wurtzite CdS, respectively, further confirming the amorphous nature of the Pd cores in heterophase nanomaterials. Importantly, the high-resolution XPS spectrum of Pd 3d revealed that Pd is not sulfurized and maintained a metallic state after the growth of CdS as the peaks located at 335.6 and 340.8 eV can be ascribed to Pd 3d3/2 and Pd 3d5/2 of zerovalent Pd, respectively (FIG. 4B). The XPS peaks located at 162.9 and 161.7 eV can be ascribed to S 2p1/2 and S 2p3/2 of CdS, respectively, indicating no PdS is formed (FIG. 4C). The metallic nature of Pd in aPd-cCdS is also confirmed by EXAFS spectra of Pd K-edge (FIG. 4D-4F). As shown in FIG. 4D, in the Pd K-edge, the white line intensity and XANES energy of aPd-cCdS match well with those of aPd nanocrystals and Pd foil. And in the Fourier transforms of EXAFS spectra, aPd-cCdS, aPd nanocrystals and Pd foil all show a dominant peak at approximately 2.45 Å in R space, corresponding to the Pd—Pd scattering path. The XANES spectra and Fourier transformed EXAFS spectra of aPd-cCdS icosapods are different from those of PdS, demonstrating that the Pd in aPd-cCdS is not sulfurized.

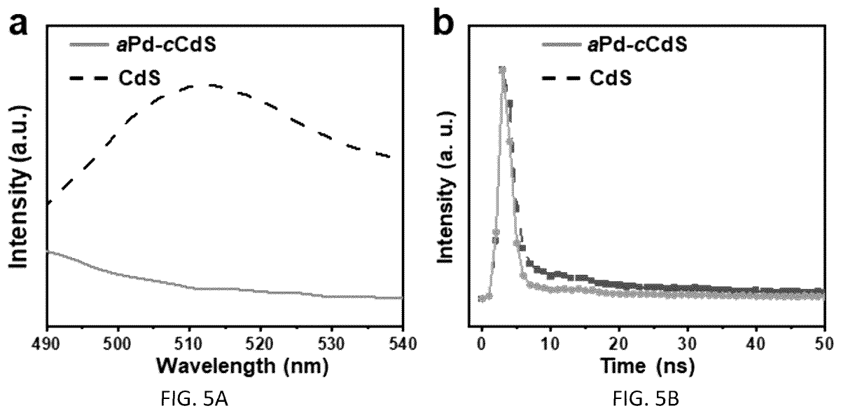

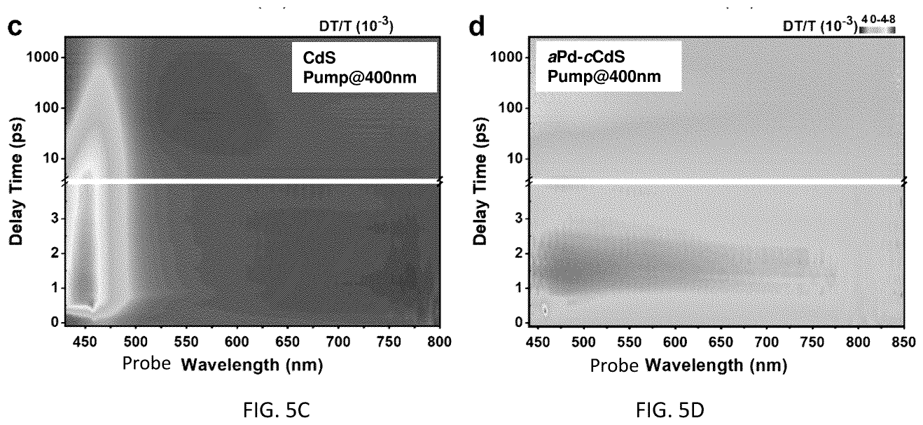

The photo-induced charge transfer process in the obtained aPd-cCdS heterophase structure is also studied systematically using photoluminescence (PL) pump-probe TA spectroscopy. From the PL spectrum (FIG. 5A), great decrease in the intensity of the PL peaks which indicated the PL quenching process in the aPd-cCdS could be observed. Moreover, in the PL lifetime spectrum, a much faster decay of aPd-cCdS is observed compared with blank CdS (FIG. 5B). The PL quenching and faster PL decay may be contributed to the electron transfer or hole quenching process.

In order to figure out the detailed charge transfer process, pump-probe TA spectroscopy is performed using 400 nm laser to excite the aPd-cCdS heterophase structures. The femtosecond transient absorption measurements are conducted in a Helios spectrometer (Ultrafast Systems LLC) with pump and probe beams derived from a regenerative amplified Ti: Sapphire laser system (Coherent Astrella, 35 fs, 4 mJ/pulse, and 1 kHz repetition rate). The 800 nm output pulse is split into two beams with a beam splitter. One beam passed through a tunable optical parametric amplifier (OperA solo, Coherent) to generate a tunable visible pump. During the measurement, the pump beam is chopped by a synchronized chopper to 500 Hz. The other beam is attenuated and focused on a CaF₂ window to generate the white light continuum with a wavelength range from 350 nm to 800 nm, referred to as the probe beam. The probe beam is focused into a 1-mm path length quartz cuvette (Starna) containing the sample in an organic solvent (e.g., toluene). The transmission of the probe is collected by a fiber optics-coupled multichannel spectrometer with complementary metal-oxide-semiconductor (CMOS) sensors and detected at a frequency of 1 kHz (Ultrafast systems, Helios). The delay between the pump and probe pulses is controlled by a motorized delay stage. Samples in 1-mm cuvettes are used for all spectroscopy measurements and stirred vigorously during the measurements.

As the 400 nm laser is over the bandgap of CdS, the CdS in the aPd-cCdS is excited. The excited carriers are followed by electron transfer to noble metal and/or self-decay. The TA spectrum of pure CdS and aPd-cCdS, displayed as a two-dimensional pseudo-color plot in FIG. 5C and FIG. 5D. The distinct bleaching bands centered at ˜480 nm that can be attributed to the bleach of the CdS 1E band. Compared with blank CdS, the aPd-cCdS exhibit much faster decay as the bleach of the CdS 1E band are decreased to none within 100 ps, while the bleach of the CdS 1E band in CdS could last more than 3000 ps. From the probed kinetics at 480 nm, the lifetime of photo-induced electron in aPd-cCdS is found to be only 124 ps, while the lifetime of photo-induced electron in CdS is more than 3000 ps. The much shorter lifetime of photo-induced electron indicated the photo-induced electron is transferred from CdS to aPd. The aPd could efficiently capture photo-induced electrons and separate the photo-induced electron and photo-induced holes.

### Photocatalytic C—N Coupling Reactions

In the third aspect of the present invention, the synthesized amorphous Pd-crystalline CdS heterostructure nanoparticles may be used as photocatalysts in a photocatalytic C—N coupling reaction to simultaneously produce hydrogen and high value-added imine.

In typical photocatalytic reactions, 10 mg aPd-cCdS heterostructures are dissolved in 5 mL an organic solvent (e.g., toluene), and then 10 mg NH₄SCN dispersing in 5 mL of N-methylformamide is added with vigorous stirring. After 10 min stirring, the aPd-cCdS heterostructures are transferred to N-methylformamide and are collected by centrifugation at 10000 rpm for 5 minutes. This process is repeated three times until the aPd-cCdS heterostructures are completely transferred into the N-methylformamide phase. The obtained solid product is washed with ethanol twice and re-dispersed in 10 mL acetonitrile. 1 mmol of organic amines, such as benzylamine, is added into 1 mg aPd-cCdS photocatalyst dispersed in 5 mL acetonitrile, and then the mixture is transferred into a 50 mL double-walled glass reactor. The suspension is thoroughly degassed by three cycles and backfilled with nitrogen and kept at a constant temperature (25° C.) with water circulated through a thermostat. The reactor is irradiated from the top through a quartz window with 300 W Xe lamp (Microsolar 300, Perfect Light) equipped with solar simulator filter with slight stirring. The light intensity is measured with a power meter (PM100D, THORLABS). The evolved hydrogen is sampled periodically by a gas chromatography (SHIMADZU, Nexis GC-2030) equipped with a thermal conductive detector using argon as the carrier gas. The solution products are identified by gas chromatography spectrometry (GC, Agilent 7890A-5975C with a DB-Waxetr column). The photocatalytic stability measurement procedure is analogous to that described for the photocatalytic measurement except that 5 mg aPd-cCdS heterostructures are used. The evolved hydrogen is sampled by gas chromatography every 12 hours. The apparent quantum yield (AQY) of the photocatalyst is calculated using the following formula.

$$A Q Y = \frac{{N}_{c}}{{N}_{i}} \times 100 \% = \frac{2 \times {τ}_{{H}_{2}}}{{I}_{i}} \times 100 \%$$

where N_{C} is the number of photons that are converted into products (H₂), Nᵢ is the number of incident photons, r_{H}_{₂} is the rate of H₂ generation, Iᵢ is incident light intensity.

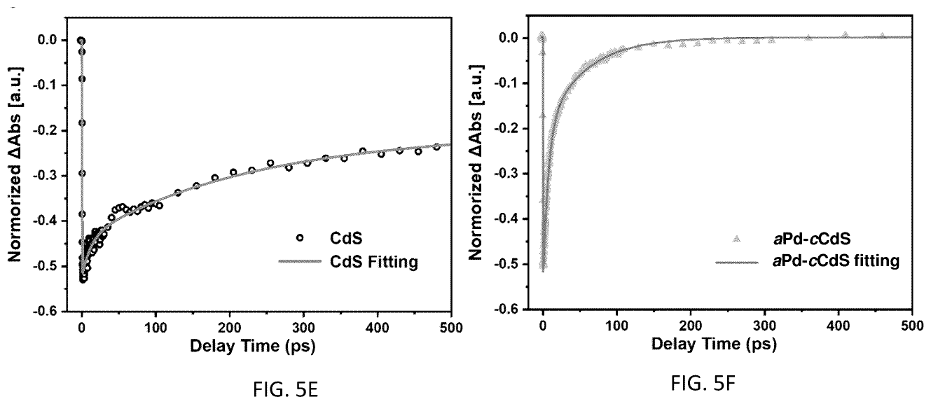

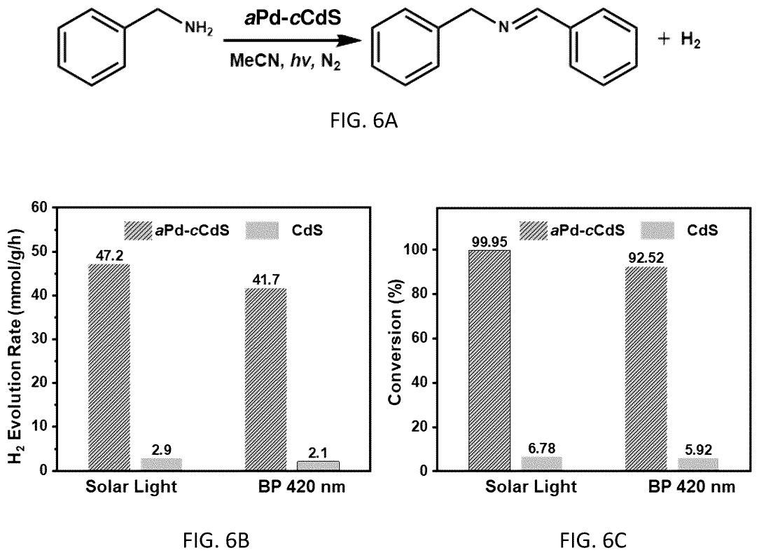

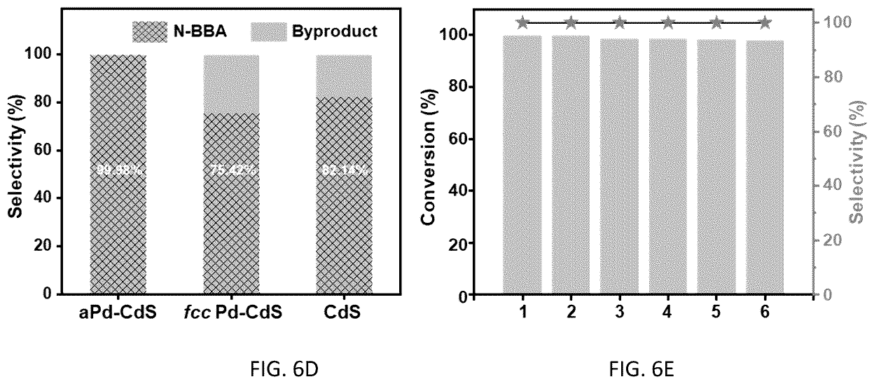

As shown in FIGS. 6A-6C, the aPd-cCdS photocatalyst could convert the benzylamine into highly valuable N-benzylbenzaldimine with a conversion rate of 46.9 mmol/g/h and the selectivity of more than 99.99%. Importantly, the aPd-cCdS photocatalyst could produce clean energy (H₂) with an evolution rate of 49.9 mmol/g/h at the same time, which is more than 15 times higher than CdS.

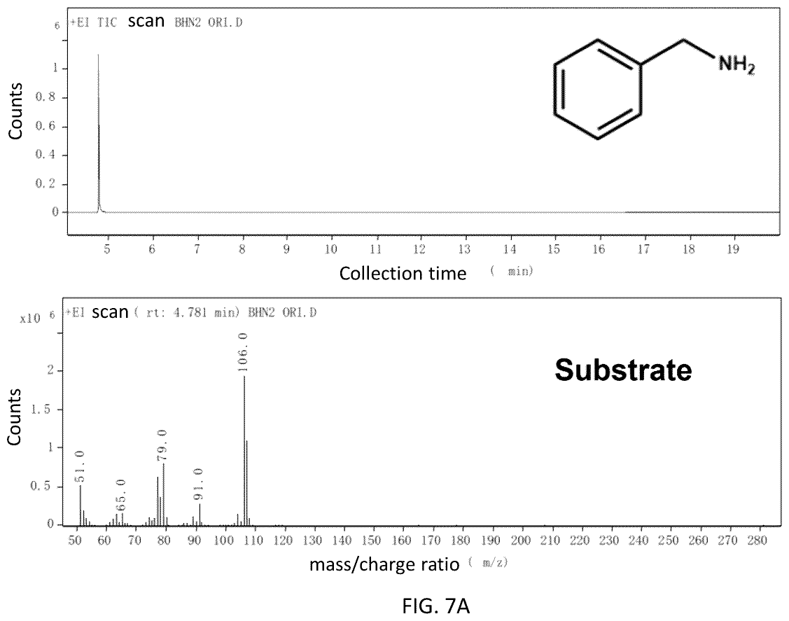

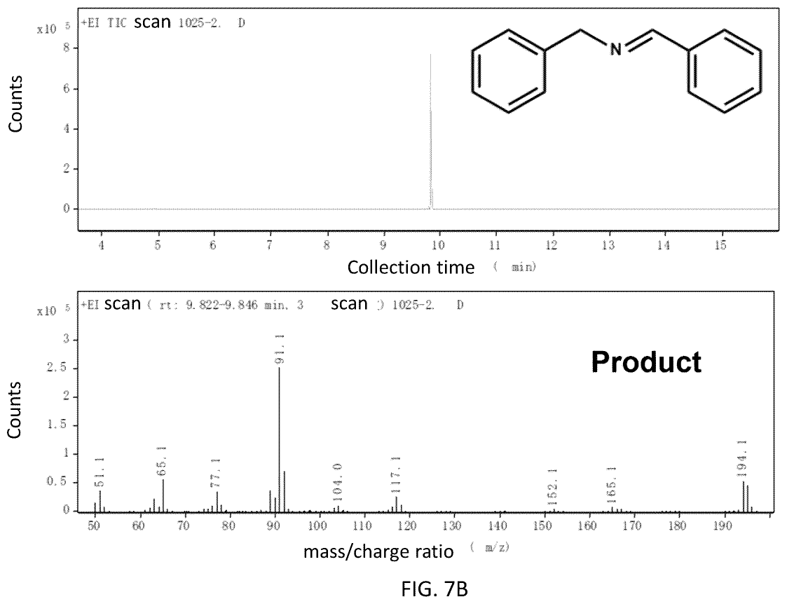

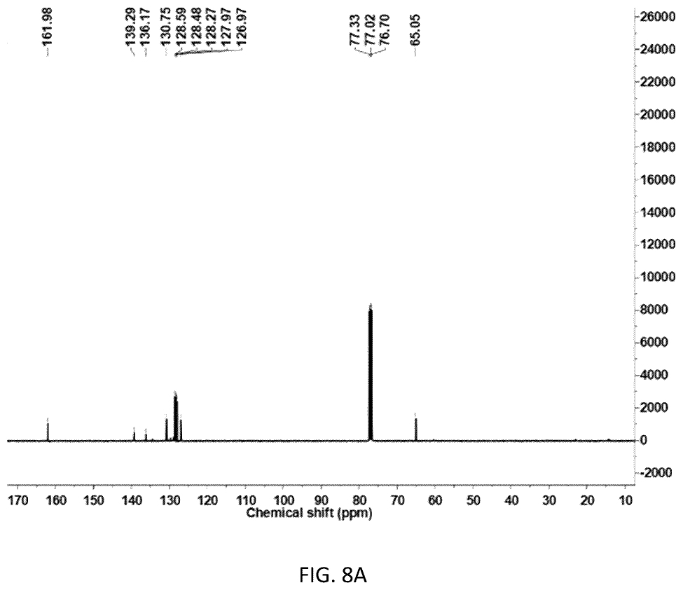

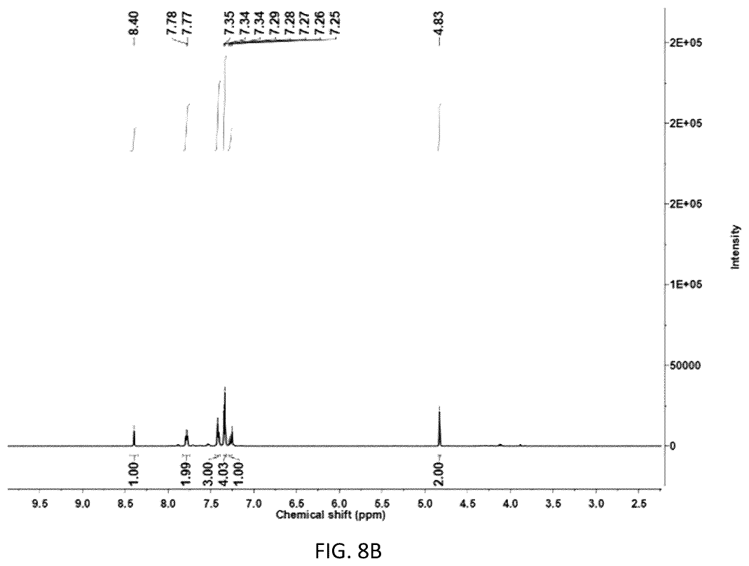

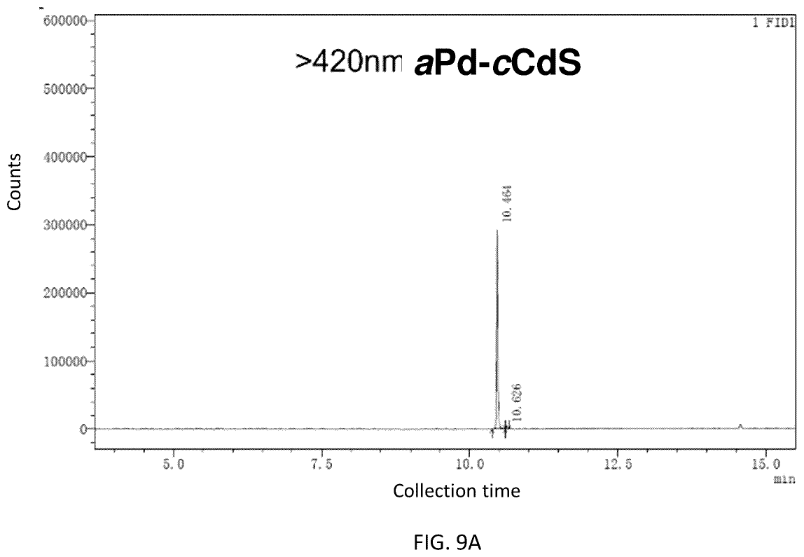

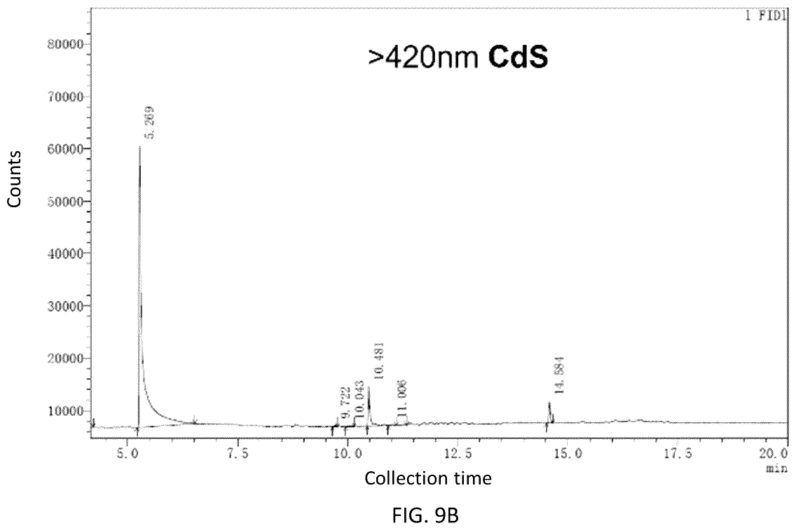

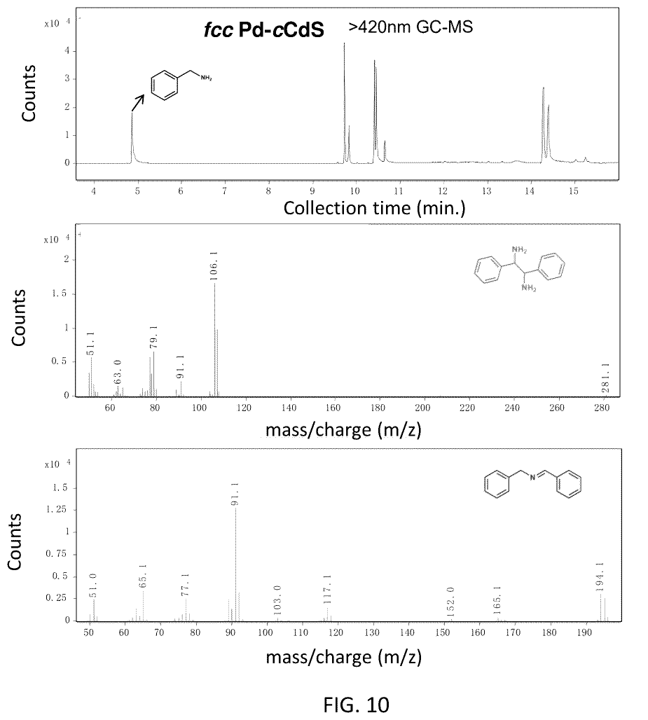

Moreover, the aPd-cCdS photocatalyst could be separated from N-benzylbenzaldimine by sample centrifuge and could be recycled more than 6 times without any decrease in performance. The AQY of aPd-cCdS photocatalyst is determined to be 31.5%. Importantly, the selectivity of the aPd-cCdS is much higher than CdS and fcc Pd-cCdS (FIGS. 6D and 6E). As shown in the gas chromatography-mass spectrometry (GC-MS) spectra (FIGS. 7A-7B), the product in the photocatalytic C—N coupling reactions is pure N-benzylbenzaldimine using aPd-cCdS heterophase structures. Referring to FIGS. 8A and 8B, the ¹H NMR (400 MHz, CDCl₃) δ [ppm] 8.40 (s, 1H), 7.78-7.77 (m, 2H), 7.35-7.34 (m, 3H), 7.29-7.27 (m, 4H), 7.26-7.25 (m, 1H), 4.83 (s, 2H) and ¹³C NMR (400 MHz, CDCl₃) δ [ppm] 161.98, 139.29, 136.17, 130.75, 128.59, 128.48, 128.27, 127.97, 126.97, 65.05 are well indexed to N-benzylbenzaldimine. However, the GC spectra of the product using aPd-cCdS or CdS (FIGS. 9A and 9B) and the corresponding MS spectra of product in photocatalytic C—N coupling reactions using fcc Pd-cCdSheterostructures (FIG. 10) show that the products in the photocatalytic C—N coupling reactions using CdS or fcc Pd-cCdS are a mixture of several imines. This result indicates the superiority of our amorphous-crystalline heterophase structures, which could exhibit higher performance and selectivity than the components and crystalline counterparts as shown in Table 1.

TABLE 1
Summary of the performance of aPd-cCdS heterostructures
and CdS in photocatalytic C—N coupling reactions

| | | | | Yeild | Selectivity | H₂ | Reaction |
|---|---|---|---|---|---|---|---|
| Entry | Catalyst | Condition | | (%) | (%) | (umol) | time (h) |
| 1 | a-Pd-cCdS | >420 | nm | 97.59 | 99.93 | 95.07 | 2 |
| 2 | CdS | >420 | nm | 6.78 | 73.89 | 0.868 | 2 |
| 3 | Q-Pd | >420 | nm | 0 | 0 | 0 | 2 |
| 4 | a-Pd-cCdS | BP 420 | nm | 89.78 | 99.25 | 85.56 | 2 |
| 5 | CdS | BP 420 | nm | 6.62 | 75.14 | 0.256 | 2 |
| 6 | a-Pd | BP 420 | nm | 0 | 0 | 0 | 2 |

The aPd-cCdS photocatalyst also presents the world's best activity and selectivity in the photosynthetic reactions (Table 2), indicating the integration of the superior catalytic performance of amorphous noble metal nanomaterials, efficient solar energy conversion ability of semiconductors, as well as the synergistic effect between them, it is believed that the controlled construction of amorphous noble metal-crystalline semiconductor heterostructures could be a promising route to the development of high performance catalysts towards photocatalytic reactions.

TABLE 2
Comparison of the performance of aPd-cCdS heterostructures with previously
reported materials in photocatalytic C—N coupling reactions

| | H₂ Evolution | | | Reaction | | | |
|---|---|---|---|---|---|---|---|
| Catalyst | Rate | | Reactions | conditions | Yield | Selectivity | Light Source |
| aPd-cCdS | 47.2 | mmol/g/h | C—N coulping | 2 mg catalyst; Ar | 2 h 99.9% | >99.99% | 300 W Xe lamp >420 nm |
| CdS/Single Pd-Sx | 11.8 | mmol/g/h | C—N coulping | 10 mg catalyst; Ar | 6 h 99.99% | >98.99 | 300 W Xe lamp >420 nm |
| Ni/CdS | 15.9 | mmol/g/h | C—N coulping | 2 mg CdS; Ar | ~96% | >99% | 300 W Xe lamp >420 nm |
| Pd-CdS/SiO₂ | 22.8 | mmol/g/h | C—N coulping | 10 mg catalyst; Ar | 6 h 99.0% | >99% | 300 W Xe lamp >420 nm |
| g-C₃N₄—Pd | ~4.2 | mmol/g/h | C—N coulping | 50 mg catalyst; Ar | 8 h 96.0% | >99% | 300 W Xe lamp >420 nm |
| WS₂-Fd | N.A. | | C—C coulping | 10 mg catalyst; Ar | 3 h ~80% | >99% | 80 W White LED lamp |
| CdS/Pd single atom | 89.1 | mmol/g/h | C—C coulping | 10 mg catalyst; Ar | 35.1 mmol/g/h | ~100% | 2000 W Xe lamp >420 nam |

Synthesis and Characterization of aPd-cCu₂₋ₓS Heterophase Nanoparticles.

In a typical synthesis of aPd-cCu₂₋ₓS, 10 mg of CuCl₂, 1 mL of an organic solvent (e.g., oleylamine), 6 mL of 1-octadecene are added into a 50 mL three-neck flask and degassed at 100° C. for 30 min under vigorous magnetic stirring at 750 r.p.m. After it is heated to 180° C. under N₂ atmosphere, the solution becomes yellow. A mixture of 2 mg of aPd seeds, 6 mg of NH₄SCN, 1.5 mL of an organic solvent (e.g., oleylamine) is injected into the flask in a constant injection rate ranging from 1 to 10 mL/h, and the temperature is kept at 170-180° C. for 1 to 3 min. Then the reaction is stopped by removing the heating mantle. After the solution is cooled down to 100° C., 5 mL of an organic solvent (e.g., toluene) is injected into the reaction flask. Then, 5 mL of ethanol is added to the solution, and the product is collected by centrifuge at 8,000 r.p.m. for 3 min. The obtained precipitate is washed with a mixture of organic solvent (e.g., toluene and ethanol (v/v=1/1)) and then dispersed into 10 mL of an organic solvent (e.g., toluene).

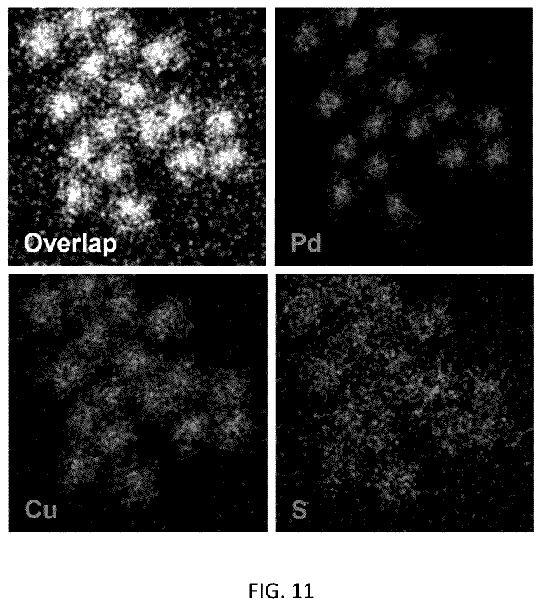

The HAADF-STEM image and the corresponding EDS mapping as shown in FIG. 11 confirms the controlled growth of the aPd-cCu₂₋ₓS heterostructures is achieved.

Synthesis and Characterization of aPd-cNi₂S₃ Heterophase Nanoparticles

In a typical synthesis of aPd-cNi₂S₃. 10 mg of Ni(acac)₂, 1 mL of an organic solvent (e.g., oleylamine), 6 mL of 1-octadecene are added into a 50 mL three-neck flask and degassed at 100° C. for 30 min under vigorous magnetic stirring at 750 r.p.m. After it is heated to 160° C. under N₂ atmosphere, the solution becomes green. A mixture of 2 mg of aPd seeds, 6 mg of NH₄SCN, 1.5 mL of an organic solvent (e.g., oleylamine) is injected into the flask in a constant injection rate ranging from 1 to 10 mL/h, and the temperature is kept at 160° C. for 4 to 10 min. Then the reaction is stopped by removing the heating mantle. After the solution is cooled down to 100° C., 5 mL of an organic solvent (e.g., toluene) is injected into the reaction flask. Then, 5 mL of ethanol is added to the solution, and the product is collected by centrifuge at 8,000 r.p.m. for 3 min. The obtained precipitate is washed with a mixture of organic solvents (e.g., toluene and ethanol (v/v=1/1)) and then dispersed into 10 mL of an organic solvent (e.g., toluene).

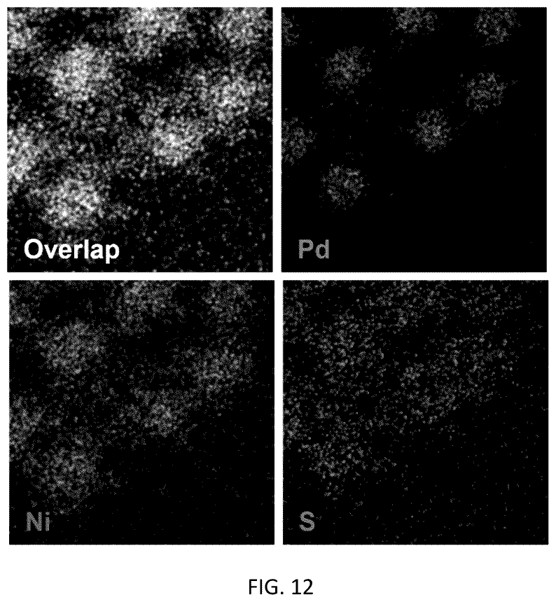

The HAADF-STEM image and the corresponding EDS mapping as shown in FIG. 12 confirms that the controlled growth of the aPd-cNi₂S₃ heterostructures is achieved.

In the second aspect of the present invention, a robust and general wet-chemical synthetic method is provided to synthesize amorphous noble metal-crystalline metal heterophase nanoparticles having an amorphous noble metal core and a crystalline metal shell. The provided method comprises: dispersing amorphous noble metal-based nanoparticle seeds into an organic solvent (e.g., oleylamine) to form a first mixture; degassing the first mixture at room temperature; preheating the first mixture under nitrogen (N₂) atmosphere at a preheat temperature for a preheat time under magnetic stirring; dissolving a metal precursor in an organic solvent (e.g., oleylamine) to form a second mixture; injecting the second mixture into the first mixture to form a third mixture at a constant injection rate; keeping the third mixture at a growth temperature for a growth time to form the noble metal-based amorphous-crystalline metal heterophase nanoparticles.

In accordance with some embodiments of the present invention, the provided method is used to synthesize amorphous Pd-crystalline metal heterostructures and comprises the preparation of aPd nanoparticle seeds, the reduction of the metal compound (such as Au or Ag compound) precursor with oleylamine as both the solvent and reductant, and the growth of crystalline metal on aPd seeds to form amorphous Pd-crystalline Au (aPd-cAu) heterophase nanoparticles or amorphous Pd-crystalline Ag (aPd-cAg) heterophase nanoparticles.

Synthesis and Characterization of aPd-cAu Heterophase Nanoparticles

In a typical synthesis of aPd-cAu, 0.2 mg of the as-prepared amorphous Pd nanoparticles are dispersed into 2 mL of an organic solvent (e.g., oleylamine) in a 50 mL Schlenk tube and sonicated at room temperature to ensure complete dissolution. After being sealed with a rubber plug, the tube is evacuated for 10 min at room temperature and purged with N₂ gas. Subsequently, the bottle is pre-heated at 100° C. under magnetic stirring for 10 min. 1 mg of HAuCl₄·xH₂O dissolved in 1 mL of an organic solvent (e.g., oleylamine) is injected into the above-mentioned reaction solution using a syringe pump with a rate of 1 mL/h. After reaction at 100° C. for 1 hour, the product is collected by centrifugation at 10,000 rpm for 1 min. Then the as-obtained products are re-dispersed into 5 mL of an organic solvent (e.g., toluene) and sonicated for 1 min, followed by adding 5 mL of ethanol to precipitate them. The product is then collected by centrifugation at 10,000 rpm for 1 min. The washing process is repeated twice, and finally, the products are re-dispersed in an organic solvent (e.g., toluene).

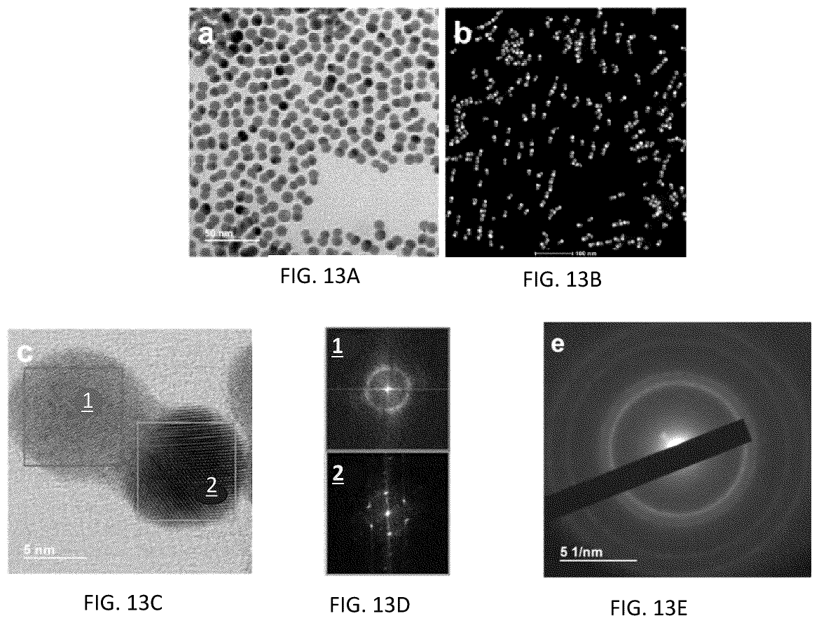

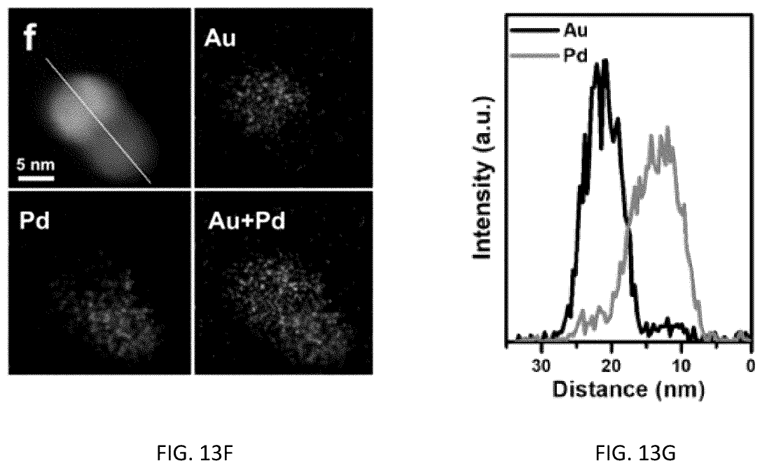

As revealed by low-magnification TEM (FIG. 13A) and STEM (FIG. 13B) images, crystalline face-centered cubic (fcc) Au is grown on amorphous Pd nanoparticles to form aPd-cAu heterostructure which has a very uniform size and morphology, with an average size of 20 nm. From high-resolution TEM image (FIG. 13C), the crystalline-amorphous heterophase structure can be clearly observed, with the size of amorphous domain (area “1” in FIG. 13C) and crystalline domain (area “2” in FIG. 13C) almost the same. The amorphous and fcc phases of the two domains are further confirmed by the diffused ring and bright spots in the corresponding FFT patterns shown in FIG. 13D, respectively. From SAED pattern (FIG. 13E), diffraction rings corresponding to the fcc phase could be observed, confirming the formation of amorphous-fcc heterophase. The elemental distribution of aPd-cAu is investigated by EDS mapping (FIG. 13F) and line scan (FIG. 13G), the separation of Au and Pd element is clear as observed in EDS mapping and line scan, the crystalline part is mainly Au while the amorphous part mainly composed of Pd, the Au content is about 47% by EDS spectrum.

Synthesis and Characterization of aPd-cAg Heterophase Nanoparticles

In a typical synthesis of aPd-cAg, 0.2 mg of the as-prepared amorphous Pd nanoparticles are dispersed into 2 mL of an organic solvent (e.g., oleylamine) in a 50 mL Schlenk tube and sonicated at room temperature to ensure complete dissolution. After being sealed with a rubber plug, the tube is evacuated for 10 min at room temperature and purged with N₂ gas. Subsequently, the bottle is pre-heated at 160° C. under magnetic stirring for 10 min. 1 mg of AgNO₃ dissolved in 1 mL of an organic solvent (e.g., oleylamine) is injected into the above-mentioned reaction solution using a syringe pump with a rate of 1 mL/h. After reaction at 160° C. for 1 hour, the product is collected by centrifugation at 10,000 rpm for 1 min. Then the as-obtained products are re-dispersed into 5 mL of an organic solvent (e.g., toluene) and sonicated for 1 min, followed by adding 5 mL of ethanol to precipitate them. The product is then collected by centrifugation at 10,000 rpm for 1 min. The washing process is repeated twice, and finally, the products are re-dispersed in an organic solvent (e.g., toluene).

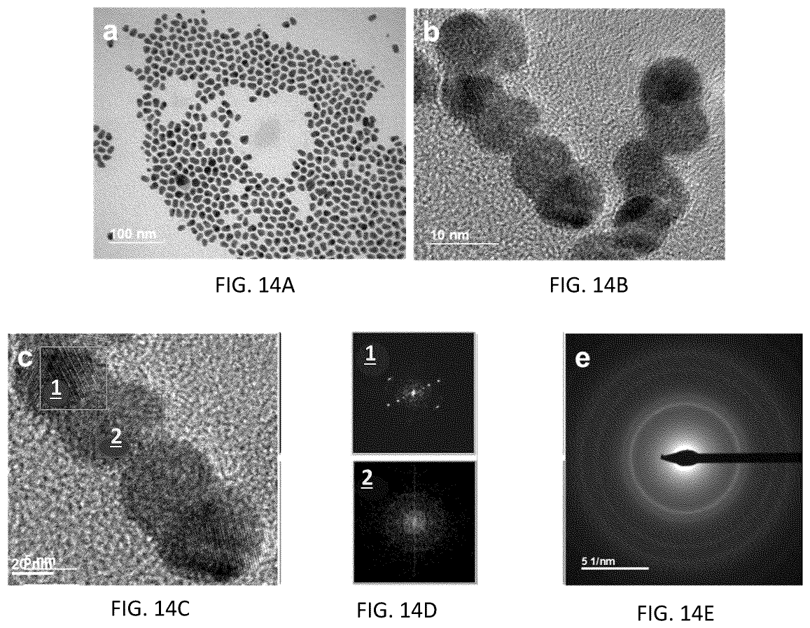

As revealed by TEM image in FIGS. 14A-14C, the aPd-cAg possesses a similar structure with that of aPd-cAu. The amorphous-crystalline heterophase is obvious and interface between crystalline (area “2” in FIG. 14C) and amorphous domains (area “1” in FIG. 14C) could be seen. The crystalline side should be fcc phase according to the FFT pattern (area “2” in FIG. 14D), the amorphous phase in the one side of aPd-cAg can be confirmed by the corresponding FFT pattern (area “1” in FIG. 14D). The SAED pattern (FIG. 14E) further confirms that amorphous-crystalline heterostructure is formed.

The embodiments may be chosen and described in order to best explain the principles of the invention and its practical application, thereby enabling others skilled in the art to understand the invention for various embodiments and with various modifications that are suited to the particular use contemplated. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations. While the apparatuses disclosed herein have been described with reference to particular structures, shapes, materials, composition of matter and relationships . . . etc., these descriptions and illustrations are not limiting. Modifications may be made to adapt a particular situation to the objective, spirit and scope of the present disclosure. All such modifications are intended to be within the scope of the claims appended hereto.

What is claimed is:

1. A method for synthesizing amorphous noble metal-crystalline semiconductor heterophase nanoparticles, each having an amorphous noble metal core and a crystalline semiconductor shell, the method comprising:
mixing amorphous noble metal-based nanoparticle seeds, chalcogen and a first solvent to form a first mixture;
mixing a metal precursor, fatty acid and a second solvent to form a second mixture;
degassing the second mixture at a degassing temperature for a degassing time under magnetic stirring;
heating the second mixture to a first temperature under nitrogen (N₂) atmosphere;
cooling the second mixture to a second temperature;
injecting the first mixture into the second mixture to form a third mixture;
keeping the third mixture at a growth temperature for a growth time to form the amorphous noble metal-crystalline semiconductor heterophase nanoparticles.

2. The method of claim 1, wherein
the amorphous noble metal-based nanoparticle seeds are amorphous palladium (Pd)-based nanoparticle seeds;
the chalcogen is ammonium thiocyanate (NH₄SCN); and
a weight ratio of the amorphous Pd-based nanoparticle seeds to the NH₄SCN is 1:3.

3. The method of claim 2, wherein the metal precursor includes one or more cadmium (Cd)-based compounds such that the core is constructed of amorphous Cd and the shell is constructed of crystalline cadmium sulphide (CdS).

4. The method of claim 3, wherein the one or more Cd-based compounds include cadmium oxide (CdO) and cadmium chloride (CdCl₂).

5. The method of claim 4, wherein weight ratios of amorphous Pd-based nanoparticle seeds to the CdO and CdCl₂ are 1:6 and 10:9 respectively.

6. The method of claim 2, wherein the metal precursor includes one or more nickel (Ni)-based compounds such that the core is constructed of amorphous Pd and the shell is constructed of crystalline nickel sulphide (Ni₂S₃).

7. The method of claim 6, wherein the one or more Ni-based compounds include nickel(II) bis(acetylacetonate) (Ni(acac)₂).

8. The method of claim 7, wherein a weight ratio of the amorphous Pd-based nanoparticle seeds to the Ni(acac)₂ is 1:5.

9. The method of claim 2, wherein the metal precursor includes one or more copper (Cu)-based compounds such that the core is constructed of amorphous Pd and the shell is constructed of crystalline copper sulphide (Cu₂-xS).

10. The method of claim 9, wherein the one or more Cu-based compounds include copper (II) chloride (CuCl₂).

11. The method of claim 10, wherein a weight ratio of the amorphous Pd-based nanoparticle seeds to the CuCl₂ is 1:5.

12. A method for synthesizing amorphous noble metal-crystalline metal heterophase nanoparticles, each having a Janus structure with an amorphous noble metal domain and a crystalline metal domain attached side by side with the amorphous noble metal domain, the method comprising:
dispersing amorphous noble metal-based nanoparticle seeds into a first solvent to form a first mixture;
degassing the first mixture at room temperature;
preheating the first mixture under nitrogen (N₂) atmosphere at a preheat temperature for a preheat time under magnetic stirring;
dissolving a metal precursor in a second solvent to form a second mixture;
injecting the second mixture into the first mixture to form a third mixture at a constant injection rate;
keeping the third mixture at a growth temperature for a growth time to form the amorphous noble metal-crystalline metal heterophase nanoparticles.

13. The method of claim 12, wherein the amorphous noble metal-based nanoparticle seeds are amorphous palladium (Pd)-based nanoparticle seeds.

14. The method of claim 13, wherein the metal precursor includes one or more gold (Au)-based compounds such that a Janus structure with an amorphous noble metal domain and a crystalline metal domain attached side by side with the amorphous noble metal domain is obtained.

15. The method of claim 14, wherein the one or more Au-based compounds include hydrogen tetrachloroaurate(III) (HAuCl₄·xH₂O).

16. The method of claim 15, wherein a weight ratio of the amorphous Pd-based nanoparticle seeds to the HAuCl₄·xH₂O is 1:5.

17. The method of claim 13, wherein the metal precursor includes one or more silver (Ag)-based compounds such that a Janus structure with an amorphous noble metal domain and a crystalline metal domain attached side by side with the amorphous noble metal domain is obtained.

18. The method of claim 17, wherein the one or more Ag-based compounds include silver nitrate (AgNO₃).

19. The method of claim 18, wherein a weight ratio of the amorphous Pd-based nanoparticle seeds to the AgNO₃ is 1:5.

20. A method of using amorphous Pd-crystalline CdS heterostructure nanoparticles as photocatalysts in a photocatalytic C—N coupling reaction to produce hydrogen and an imine, comprising:
dissolving the amorphous Pd-crystalline CdS heterostructure nanoparticles in an organic solvent to form a first mixture;
mixing NH₄SCN in N-methylformamide to form a second mixture;
dispersing the second mixture into the first mixture with vigorous stirring to transform the amorphous Pd-crystalline CdS heterostructure nanoparticles to a solid product with a N-methylformamide phase;
washing the solid product with ethanol;
dispersing the washed solid product in acetonitrile to form a third mixture;
adding an amine into the third mixture to form a fourth mixture;
degassing the fourth mixture;
keeping the degassed fourth mixture at room temperature under nitrogen (N₂) atmosphere;
irritating the fourth mixture with a 300 W Xe lamp to produce the hydrogen and convert the amine into the imine.

21. The method of claim 20, wherein the amine is a benzylamine and the imine is a N-benzylbenzaldimine.
